(19) United States
(12) Patent Application Publication
(10) Pub. No.: US 2019/0177282 A1
(43) Pub. Date: Jun. 13, 2019

(54) A PROCESS FOR THE PREPARATION OF ELUXADOLINE

(71) Applicant: SUN PHARMACEUTICAL INDUSTRIES LIMITED, Mumbai, Maharashtra (IN)

(72) Inventors: Pankaj Kumar SINGH, Gurgaon (IN); Mukesh Kumar MADHRA, Karnal (IN); Mohan PRASAD, Gurgaon (IN)

(21) Appl. No.: 16/306,350

(22) PCT Filed: May 30, 2017
(86) PCT No.: PCT/IB17/53182

§ 371 Date: Nov. 30, 2018

(30) Foreign Application Priority Data
May 30, 2016 (IN) 201611018450

(51) Int. Cl. C07D 233/64; C07C 269/06; C07C 271/00

(52) U.S. Cl. CPC C07D 233/64; C07C 271/00; C07C 269/06

(57) ABSTRACT

The present invention relates to an improved process for the preparation of eluxadoline and its intermediates and that includes the use of an aqueous surfactant solution in the preparation of N²-[(benzyloxy)carbonyl]-N-(2-oxo-2-phenylethyl)-L-alaninamide, an intermediate in the preparation of eluxadoline.

### FIELD OF THE INVENTION

The present invention relates to an improved process for the preparation of eluxadoline and its intermediates.

### BACKGROUND OF THE INVENTION

Eluxadoline chemically is 5-[[[(2S)-2-amino-3-[4-(aminocarbonyl)-2,6-dimethylphenyl]-1-oxopropyl][(1S)-1-(4-phenyl-1H-imidazol-2-yl)ethyl]amino]methyl]-2-methoxybenzoic acid, represented by Formula I.

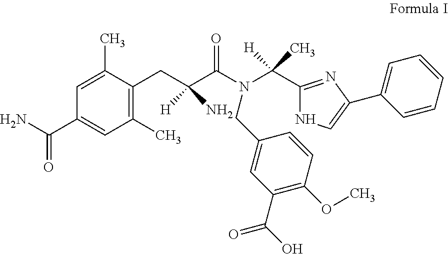 [SMILES: COc1ccc(CN(C(=O)[C@@H](N)Cc2c(C)cc(C(N)=O)cc2C)[C@@H](C)c2nc(-c3ccccc3)c[nH]2)cc1C(=O)O]

Eluxadoline is a mu-opioid receptor agonist, indicated in adults for the treatment of irritable bowel syndrome with diarrhea (IBS-D).

U.S. Pat. No. 7,741,356 describes a process for the preparation of eluxadoline comprising the step of reacting N-[(benzyloxy)carbonyl]-L-alanine of Formula III with 2-amino-1-phenylethanone hydrochloride of a salt of Formula IV in dichloromethane in the presence of N-methylmorpholine, 1-hydroxybenzotriazole and 1-[3-(dimethylamino)propyl]-3-ethylcarbodiimide hydrochloride to obtain N²-[(benzyloxy)carbonyl]-N-(2-oxo-2-phenylethyl)-L-alaninamide of Formula II.

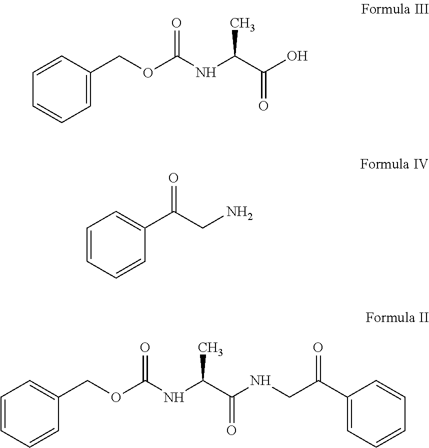 [SMILES: C[C@H](NC(=O)OCc1ccccc1)C(=O)O ; NCC(=O)c1ccccc1 ; C[C@H](NC(=O)OCc1ccccc1)C(=O)NCC(=O)c1ccccc1]

There is a need in the art to develop an improved process for the preparation of eluxadoline and its intermediates.

### SUMMARY OF THE INVENTION

The present invention relates to an improved process for the preparation of eluxadoline and its intermediates.

The present invention provides an environmentally friendly, cost-effective, and industrially advantageous process for the preparation of eluxadoline and its intermediates. The process of the present invention involves the use of an aqueous surfactant solution for the preparation of pure N²-[(benzyloxy)carbonyl]-N-(2-oxo-2-phenylethyl)-L-alaninamide of Formula II, an intermediate of eluxadoline. The aqueous surfactant solution is beneficial for a large scale synthesis of pure N²-[(benzyloxy)carbonyl]-N-(2-oxo-2-phenylethyl)-L-alaninamide of Formula II, owing to its inexpensive nature, ease of handling, and ease of isolation. Further, the process of the present invention avoids the use of column chromatography. An additional advantage of the present invention is that it involves the use of water as a solvent.

### DETAILED DESCRIPTION OF THE INVENTION

The term “about,” as used herein, refers to any value which lies within the range defined by a number up to +10% of the value.

The term “room temperature,” as used herein, refers to the temperature in the range of 25° C. to 35° C.

The term “pure,” as used herein, refers to N²-[(benzyloxy)carbonyl]-N-(2-oxo-2-phenylethyl)-L-alaninamide of Formula II, having a chromatographic purity of greater than or equal to about 80%, preferably, a chromatographic purity of greater than or equal to about 85%, preferably, a chromatographic purity of greater than or equal to about 95% and more preferably, a chromatographic purity of greater than or equal to about 99%.

The term “nitrogen protecting group,” as used herein, refers to acetyl, tert-butoxycarbonyl, trityl, p-toluenesulfonyl, benzyloxy carbonyl, or 9-fluorenylmethoxy carbonyl groups.

The term “surfactant,” as used herein, refers to a surface active agent or a mixture of agents that lower the interfacial tension between a solid and a liquid, or two liquids. The surfactant is a physiologically acceptable anionic, cationic, or nonionic surfactant.

Examples of anionic surfactants include, but are not limited to, sodium oleyl isethionate, sodium salt of oleyl methyl tauride, palmito nitrile sulfonate, sodium alpha naphthalene monosulfonate, sodium octahydro anthracene sulfonate, potassium dodecyl sulfate, and ammonium dodecyl sulfate.

Examples of cationic surfactants include, but are not limited to, dodecylamine acetate, octadecylamine acetate, 2-undecylimidazoline, oleylaminodiethylamine, dioctadecyl dimethyl ammonium chloride, didodecyl dimethyl ammonium chloride, dihexadecyl dimethyl ammonium chloride, beta-hydroxyethylsterarylamide, oleylbenzylaminoethylene diethylamine hydrochloride, methylheptadecyl benzimidazol hydrobromide, cetylpyridinium chloride, octadecylsulfonium methyl sulfate, and octadecylchloromethyl ether.

Examples of commercially available, nonionic surfactants include, but are not limited to, Tergitol® TMN-6, Tergitol® 15S40, Tergitol® 15S7, Brij®-35, Triton® X-100, TPGS-750M, PTS-7, and SPGS-550M.

A first aspect of the present invention provides a process for the preparation of a compound of Formula II,

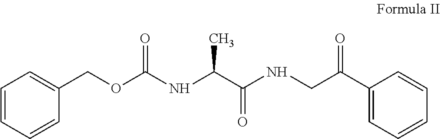 [SMILES: C[C@H](NC(=O)OCc1ccccc1)C(=O)NCC(=O)c1ccccc1]

comprising coupling a compound of Formula III

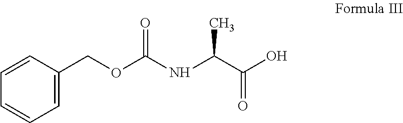 [SMILES: C[C@H](NC(=O)OCc1ccccc1)C(=O)O]

with a compound of Formula IV or a salt thereof

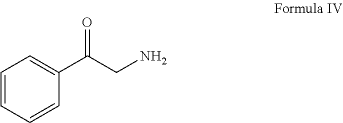

in the presence of an aqueous surfactant solution.

A second aspect of the present invention provides a process for the preparation of eluxadoline of Formula I,

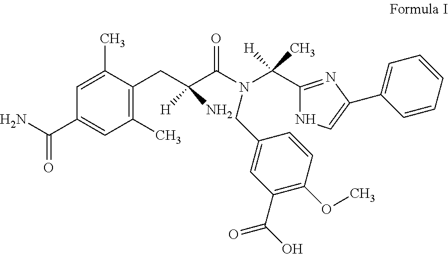 [SMILES: COc1ccc(CN(C(=O)[C@@H](N)Cc2c(C)cc(C(N)=O)cc2C)[C@@H](C)c2nc(-c3ccccc3)c[nH]2)cc1C(=O)O]

comprising

a) coupling a compound of Formula III

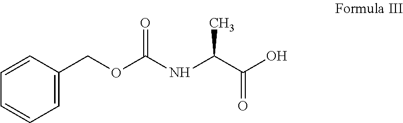 [SMILES: C[C@H](NC(=O)OCc1ccccc1)C(=O)O]

with a compound of Formula IV or a salt thereof

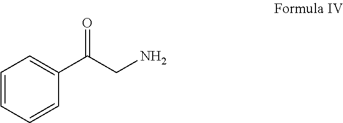

in the presence of an aqueous surfactant solution to obtain a compound of Formula II; and

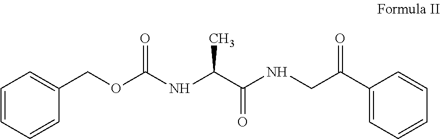 [SMILES: C[C@H](NC(=O)OCc1ccccc1)C(=O)NCC(=O)c1ccccc1]

b) converting the compound of Formula II to eluxadoline of Formula I.

The coupling of the compound of Formula III with the compound of Formula IV or a salt thereof to obtain the compound of Formula II is carried out in the presence of an aqueous surfactant solution, a coupling agent, and a base.

The coupling agent is selected from the group consisting of N,N-dicyclohexylcarbodiimide (DCC), 1-ethyl-3-(3-dimethylaminopropyl) carbodiimide (EDCI), 1,1′-carbonyldiimidazole (CDI), N,N,N′,N-tetramethyl-O-(benzotriazol-1-yl)uronium tetrafluoroborate (TBTU), N,N,N′,N′-tetramethyl-O-(1H-benzotriazol-1-yl)uronium hexafluorophosphate (HBTU), and (1-cyano-2-ethoxy-2-oxoethylidenaminooxy) dimethylamino-morpholino-carbenium hexafluorophosphate (COMU).

The base is selected from the group consisting of triethylamine, N-methylmorpholine, 4-(N,N-dimethylamino)pyridine, 2,6-lutidine, 1-methylpiperidine, N-ethyldiisopropylamine, N,N-diisopropylethylamine, 2,4,6-trimethylpyridine, and 2,4,6-collidine.

The coupling of the compound of Formula III with the compound of Formula IV or a salt thereof is carried out for about 4 hours to about 15 hours, for example, from about 5 hours to about 12 hours.

The coupling of the compound of Formula III with the compound of Formula IV or a salt thereof is carried out at a temperature of from about 20° C. to about 70° C., for example, from about 25° C. to about 60° C.

The compound of Formula II may optionally be isolated by filtration, decantation, extraction, distillation, evaporation, chromatography, precipitation, concentration, crystallization, centrifugation, and recrystallization. The compound of Formula II may be dried using conventional techniques, for example, drying, drying under vacuum, spray drying, air drying, or agitated thin film drying.

The compound of Formula II is converted to eluxadoline of Formula I by processes known in the art, for example, as disclosed in U.S. Pat. No. 7,741,356.

While the present invention has been described in terms of its specific aspects and embodiments, certain modifications and equivalents will be apparent to those skilled in the art, and are intended to be included within the scope of the present invention.

### Method

Chromatographic purity of the samples was determined by HPLC using Water® Alliance® HPLC system, Water 2695 separation module with 2489 UV visible detector.

The following examples are for illustrative purposes only and should not be construed as limiting the scope of the invention in any way.

### EXAMPLES

### Comparative Example: Preparation of N²-[(benzyloxy)carbonyl]-N-(2-oxo-2-phenylethyl)-L-alaninamide (Formula II) in the Absence of Surfactant

De-ionized water (DI water) (50 mL) was added to a mixture of N-[(benzyloxy)carbonyl]-L-alanine (2 g; Formula III) and 2-amino-1-phenylethanone hydrochloride (1.98 g; a salt of Formula IV) at room temperature. N-Methylmorpholine (1.1 g) was added to the reaction mixture over a period of 10 minutes to 15 minutes and then stirred for 10 minutes. 1-Ethyl-3-(3-dimethylaminopropyl)carbodiimide (1.6 g) was added to the reaction mixture and stirred for 6 hours to 7 hours at room temperature. The reaction mass was filtered, washed with DI water and then dried in an air oven at 45° C. to 50° C. to obtain the title compound.

Yield: 1.2 g

Chromatographic purity: 57.90%

### Example: Preparation of N²-[(benzyloxy)carbonyl]-N-(2-oxo-2-phenylethyl)-L-alaninamide (Formula II)

Method A:

An aqueous solution of Triton® X-100 (5 g in 50 mL de-ionised (DI) water) was added to a mixture of N-[(benzyloxy)carbonyl]-L-alanine (5 g; Formula III) and 2-amino-1-phenylethanone hydrochloride (4.97 g; a salt of Formula IV) at room temperature. The reaction mixture was stirred for 10 minutes to 15 minutes. N-Methylmorpholine (2.78 g) was added to the reaction mixture over a period of 10 minutes to 15 minutes and then stirred for 10 minutes. 1-Ethyl-3-(3-dimethylaminopropyl)carbodiimide (4 g) was added to the reaction mixture and stirred for 6 hours to 7 hours at room temperature. The reaction mass was filtered, washed with DI water and then dried in an air oven at 45° C. to 50° C. to obtain the title compound.

Yield: 4.8 g

Chromatographic purity: 95.96%

Method B:

An aqueous solution of Triton® X-100 (10 g in 50 mL DI water) was added to a mixture of N-[(benzyloxy)carbonyl]-L-alanine (5 g; Formula III) and 2-amino-1-phenylethanone hydrochloride (4.97 g; a salt of Formula IV) at room temperature. The reaction mixture was stirred for 10 minutes to 15 minutes. N-Methylmorpholine (2.78 g) was added to the reaction mixture over a period of 10 minutes to 15 minutes and then stirred for 10 minutes. 1-Ethyl-3-(3-dimethylaminopropyl)carbodiimide (4 g) was added to the reaction mixture and stirred for 6 hours to 7 hours at room temperature. The reaction mass was filtered, washed with DI water and then dried in an air oven at 45° C. to 50° C. to obtain the title compound.

Yield: 4.3 g

Chromatographic purity: 96.10%

Method C:

An aqueous solution of Triton® X-100 (20 g in 50 mL DI water) was added to a mixture of N-[(benzyloxy)carbonyl]-L-alanine (5 g; Formula III) and 2-amino-1-phenylethanone hydrochloride (4.97 g; a salt of Formula IV) at room temperature. The reaction mixture was stirred for 10 minutes to 15 minutes. N-Methylmorpholine (2.78 g) was added to the reaction mixture over a period of 10 minutes to 15 minutes and then stirred for 10 minutes. 1-Ethyl-3-(3-dimethylaminopropyl)carbodiimide (4 g) was added to the reaction mixture and stirred for 6 hours to 7 hours at room temperature. The reaction mass was filtered, washed with DI water and then dried in an air oven at 45° C. to 50° C. to obtain the title compound.

Yield: 3.9 g

Chromatographic purity: 96.55%

Method D:

An aqueous solution of Triton® X-100 (10 g in 50 mL DI water) was added to a mixture of N-[(benzyloxy)carbonyl]-L-alanine (5 g; Formula III) and 2-amino-1-phenylethanone hydrochloride (4.97 g; a salt of Formula IV) at room temperature. The reaction mixture was stirred for 10 minutes to 15 minutes. N-Methylmorpholine (2.78 g) was added to the reaction mixture over a period of 10 minutes to 15 minutes and then stirred for 10 minutes. 1-Ethyl-3-(3-dimethylaminopropyl)carbodiimide (4 g) was added to the reaction mixture and stirred for 6 hours to 7 hours at 50° C. to 55° C. The reaction mass was filtered, washed with DI water and then dried in an air oven at 45° C. to 50° C. to obtain the title compound.

Yield: 3.2 g

Chromatographic purity: 82.39%

Method E:

An aqueous solution of Triton® X-100 (2.0 g in 10 mL DI water) was added to a mixture of N-[(benzyloxy)carbonyl]-L-alanine (1 g; Formula III) and 2-amino-1-phenylethanone hydrochloride (0.994 g; a salt of Formula IV) at room temperature. The reaction mixture was stirred for 10 minutes to 15 minutes. N-Methylmorpholine (1.4 g) was added to the reaction mixture over a period of 10 minutes to 15 minutes and then stirred for 10 minutes. 1-Ethyl-3-(3-dimethylaminopropyl)carbodiimide (0.8 g) was added to the reaction mixture and stirred for 10 hours to 12 hours at room temperature. The reaction mass was filtered, washed with DI water and then dried in an air oven at 45° C. to 50° C. to obtain the title compound.

Yield: 0.75 g

Chromatographic purity: 92.92%

Method F:

An aqueous solution of Brij-35 (5 g in 50 mL DI water) was added to a mixture of N-[(benzyloxy)carbonyl]-L-alanine (5 g; Formula III) and 2-amino-1-phenylethanone hydrochloride (4.97 g; a salt of Formula IV) at room temperature. The reaction mixture was stirred for 10 minutes to 15 minutes. N-Methylmorpholine (2.78 g) was added to the reaction mixture over a period of 10 minutes to 15 minutes and then stirred for 10 minutes. 1-Ethyl-3-(3-dimethylaminopropyl)carbodiimide (4 g) was added to the reaction mixture and stirred for 10 hours to 12 hours at room temperature. The reaction mass was filtered, washed with DI water and then dried in an air oven at 45° C. to 50° C. to obtain the title compound.

Yield: 3 g

Chromatographic purity: 92.98%

Method G:

An aqueous solution of Brij-35 (10 g in 50 mL DI water) was added to a mixture of N-[(benzyloxy)carbonyl]-L-alanine (5 g; Formula III) and 2-amino-1-phenylethanone hydrochloride (4.97 g; a salt of Formula IV) at room temperature. The reaction mixture was stirred for 10 minutes to 15 minutes. N-Methylmorpholine (2.78 g) was added to the reaction mixture over a period of 10 minutes to 15 minutes and then stirred for 10 minutes. 1-Ethyl-3-(3-dimethylaminopropyl) carbodiimide (4 g) was added to the reaction mixture and stirred for 6 hours to 7 hours at room temperature. The reaction mass was filtered, washed with DI water and then dried in an air oven at 45° C. to 50° C. to obtain the title compound.

Yield: 3.2 g

Chromatographic purity: 98.47%

Method H:

An aqueous solution of TPGS-750M (2 wt. % solution in 10 mL water) was added to a mixture of N-[(benzyloxy)carbonyl]-L-alanine (2 g; Formula III) and 2-amino-1-phenylethanone hydrochloride (2.48 g; a salt of Formula IV) at room temperature. The reaction mixture was stirred for 10 minutes to 15 minutes. N-Methylmorpholine (1.1 g) was added to the reaction mixture over a period of 10 minutes to 15 minutes and then stirred for 10 minutes. 1-Ethyl-3-(3-dimethylaminopropyl)carbodiimide (1.6 g) was added to the reaction mixture and stirred for 10 hours to 12 hours at room temperature. The reaction mass was filtered, washed with DI water and then dried in an air oven at 45° C. to 50° C. to obtain the title compound.

Yield: 1.7 g

Chromatographic purity: 82.93%

Method I:

An aqueous solution of SPGS-750M (10 mL (2% w/w solution in 10 mL water)) was added to a mixture of N-[(benzyloxy)carbonyl]-L-alanine (2 g; Formula III) and 2-amino-1-phenylethanone hydrochloride (2.48 g; a salt of Formula IV) at room temperature. The reaction mixture was stirred for 10 minutes to 15 minutes. N-Methylmorpholine (1.1 g) was added to the reaction mixture over a period of 10 minutes to 15 minutes and then stirred for 10 minutes. 1-Ethyl-3-(3-dimethylaminopropyl)carbodiimide (1.6 g) was added to the reaction mixture and stirred for 10 hours to 12 hours at room temperature. The reaction mass was filtered, washed with DI water and then dried in an air oven at 45° C. to 50° C. to obtain the title compound.

Yield: 1.5 g

Chromatographic purity: 88.46%

1. (canceled)

2. A process for the preparation of eluxadoline of Formula I, 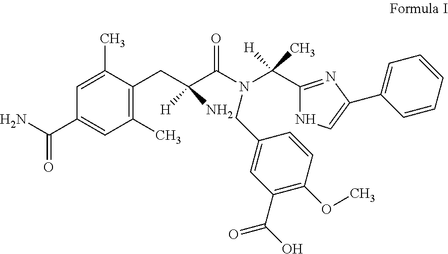 [SMILES: COc1ccc(CN(C(=O)[C@@H](N)Cc2c(C)cc(C(N)=O)cc2C)[C@@H](C)c2nc(-c3ccccc3)c[nH]2)cc1C(=O)O]
comprising:
a) coupling a compound of Formula III 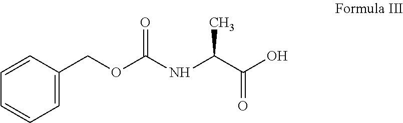 [SMILES: C[C@H](NC(=O)OCc1ccccc1)C(=O)O]
with a compound of Formula IV or a salt thereof 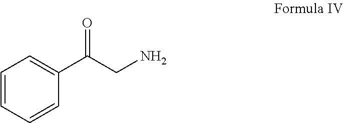
in the presence of an aqueous surfactant solution to obtain a compound of Formula II; and 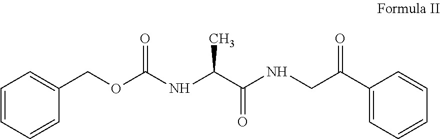 [SMILES: C[C@H](NC(=O)OCc1ccccc1)C(=O)NCC(=O)c1ccccc1]
b) converting the compound of Formula II to eluxadoline of Formula I.

4. (canceled)

5. The process according to the claim 2, wherein the coupling is carried out in the presence of a coupling agent selected from the group consisting of N,N′-dicyclohexylcarbodiimide (DCC), 1-ethyl-3-(3-dimethylaminopropyl) carbodiimide (EDCI), 1,1′-carbonyldiimidazole (CDI), N,N,N′,N′-tetramethyl-O-(benzotriazol-1-yl)uronium tetrafluoroborate (TBTU), N,N,N′,N′-tetramethyl-O-(1H-benzotriazol-1-yl)uronium hexafluorophosphate (HBTU), and (1-cyano-2-ethoxy-2-oxoethylidenaminooxy) dimethylamino-morpholino-carbenium hexafluorophosphate (COMU).

7. The process according to the claim 2, wherein the coupling is carried out in the presence of a base selected from the group consisting of triethylamine, N-methylmorpholine, 4-(N,N-dimethylamino)pyridine, 2,6-lutidine, 1-methylpiperidine, N-ethyldiisopropylamine, N,N-diisopropylethylamine, 2,4,6-trimethylpyridine, and 2,4,6-collidine.

8. The process according to claim 2, wherein the coupling is carried out in the presence of an aqueous surfactant solution prepared by contacting an anionic surfactant, a cationic surfactant, or a nonionic surfactant with water.

9. (canceled)

10. (canceled)

11. (canceled)

12. (canceled)
